SEQUENCE LISTING (6 sequences)

SEQ ID NO: 1 - protein, 25 aa, Artificial Sequence
  Specific for binding to CD20 monoclonal antibody
NTYNCEPANPSEKNSPSTQYCYSIQ

SEQ ID NO: 2 - protein, 9 aa, Artificial Sequence
  Cyclic peptide wherein the flanking cysteines form an intermolecular disulfide bond
CPYANPSLC

SEQ ID NO: 3 - protein, 9 aa, Artificial Sequence
  Cyclic peptide hwerein the flanking cysteines form an intermolecualr disulfide bond
CPYSNPSLC

SEQ ID NO: 4 - protein, 7 aa, Artificial Sequence
  Specific for binding to CD20 monoclonal antibody
WPRWLEN

SEQ ID NO: 5 - protein, 6 aa, Artificial Sequence
  Specific for binding to CD20 monoclonal antibody
WPXWLE

SEQ ID NO: 6 - protein, 12 aa, Artificial Sequence
  Specific for binding to CD20 monoclonal antibody
QDKLTQWPKWLE

(19) United States
(12) Patent Application Publication
(10) Pub. No.: US 2012/0070907 A1
(43) Pub. Date: Mar. 22, 2012

(54) METHOD TO DEPLETE MONOCLONAL ANTIBODIES IN A BIOLOGICAL SAMPLE

(76) Inventors: Mark Pescovitz, Carmel, IN (US); Benita Book, Indianapolis, IN (US); Ora Pescovitz, Ann Arbor, MI (US)

(21) Appl. No.: 12/890,288

(22) Filed: Sep. 24, 2010

Related U.S. Application Data
(63) Continuation of application No. PCT/US09/38011, filed on Mar. 24, 2009.
(60) Provisional application No. 61/038,886, filed on Mar. 24, 2008.

(51) Int. Cl. G01N 1/28; C07K 17/00

(52) U.S. Cl. 436/175; 530/300

(57) ABSTRACT

The present invention provides methods for depleting a biological sample from a patient of a humanized or chimeric monoclonal antibody prior to cross-matching the sample for transplantation. The method comprises treating the sample with a peptide or other moiety that selectively binds to the antibody, forming a peptide-antibody complex and removing the complex from the serum sample. These steps may be repeated as many times as needed to reduce the humanized or chimeric monoclonal antibody to the desired level.

### FIELD OF THE INVENTION

The present invention relates to methods for depleting antibodies present in a given biological sample from a patient. Particularly, it relates to methods for selectively depleting monoclonal antibodies from a biological sample prior to cross-matching for tissue/organ/cell transplantation.

### BACKGROUND OF THE INVENTION

Anti-lymphocyte antibodies (ALA) are used extensively in organ transplantation for the induction of immunosuppression. These antibodies may be human antibodies, humanized antibodies or chimeric antibodies. One example of such an antibody is rituximab. Rituximab, sold as Rituxan™, is a chimeric monoclonal antibody that includes about 25% murine anti-CD20 antibody 2B8, and about 75% human antibody. The Fc portion of rituximab has a human IgG1 component, which can bind to human complement, initiate antibody dependent cellular cytotoxicity (ADCC) and complement dependent cytotoxicity (CDC). Rituximab was initially developed for the treatment of B cell lymphoma, using its ability to bind to CD20 on B cells and acting by various proposed mechanisms to deplete B cells, either neoplastic or normal.

In addition to B cell lymphoma, B cells are also thought to be involved in various forms of rejection of transplanted organs including, for example, antibody mediated hyperacute rejection, which may occur within minutes of transplantation. The hyperacute rejection often results from the reactivity of pre-existing, donor specific alloantibody with the allograft. Preformed alloantibodies in an allosensitized individual react with allograft antigens present on the vascular endothelial cells, activating the classic complement pathway, ultimately resulting in rapid necrosis of the transplanted tissue. Prior allosensitization of recipients to donor alloantigens can occur by many pathways including, for example, via blood transfusion, pregnancy and from a previous graft.

Recently, rituximab has been included as part of pre-transplant conditioning regimens in addition, for example, to splenectomy and plasmapheresis, with or without intravenous immunoglobulin (IVIg), in allosensitized patients waiting for renal transplantation. There are reports of using rituximab to prevent and treat allograft rejection (both acute and chronic) and to facilitate ABO mismatched kidney transplants. If used in these situations, rituximab present in the sera from previous dosing, will interfere with future transplant immunologic monitoring. Other antibodies used for transplantation (some of which are human or humanized) include, for example, other anti-CD20 antibodies, and anti-CD3 and anti-CD52 antibodies.

Performing a cross-match test immediately before a transplant procedure is frequently critical in preventing organ rejection. Because of the increasing use of rituximab or other monoclonal antibodies prior to, or at the time of, transplant in patients, who may or may not have an alloantibody in their serum, it is important to correctly determine whether a positive cross-match is caused by the alloantibody itself, or by a lingering monoclonal, or by both.

Due to their long half-life, monoclonal antibodies such as rituximab can remain in a patient's serum for many months after the administration of the drug. The persistence of rituximab, for example, in the serum has implications for cross-matching and tissue typing analysis. Since rituximab is cytotoxic in the presence of complement, plasma/sera that include rituximab may produce a false result in cross-matching assays. The human portion of the monoclonal antibody can provide a target for the anti-human Ig fluorochromes used in flow cytometric type cross-match analysis, again, resulting in a false positive cross-match.

There are few methods in the prior art that deal with exogenous antibodies that are present in the plasma/serum that produce a false positive in a transplantation cross-match. Accordingly, it is difficult to perform transplant cross-matching analysis if exogenous antibodies are present. One method for reducing cross-matching false positives disclosed in the prior art uses pronase, a proteolytic enzyme that targets Fc receptors to remove CD20 from B cells (Bearden, C. M. et al., Human Immunology 65, 803-809 (2004)). After CD20 is removed, rituximab may not bind to B cells, making it possible to detect class I and II Major Histocompatibility (MHC) antibodies on treated B cells without causing a false positive. The concentrations of pronase required by this approach, makes this approach less than desirable because the high levels of pronase used may damage the B cell. Furthermore, this approach is not suitable reducing false positives caused by other cell surface antigens, as pronase may not be able to remove them. Another method disclosed in the prior art uses anti-mouse secondary antibody (polyclonal) conjugated to beads to titrate and remove rituximab (Bearden, C. M. et al., Journal of Immunological Methods 300, 192-199 (2005)). However, the poor specificity of anti mouse secondary antibody may result in false positive results in complement dependant cytotoxicity assays. Furthermore, this method may only be applicable to antibodies that have a murine component and would not work on humanized, or fully human or fully human-mutagenized monoclonal antibodies.

Thus, there is a need for a rapid and selective method to eliminate rituximab and other such antibodies from a biological sample before cross-matching the sample for organ transplantation as well as after transplantation. It would also be desirable if the method could be adapted for other chimeric, humanized, fully human or fully human-mutagenized monoclonal antibodies as use of monoclonal antibodies as therapeutics is expected to increase.

### SUMMARY OF THE INVENTION

In one aspect of the present invention there is provided a method of treating a biological sample from a patient for transplantation cross-matching, wherein the patient has been administered a monoclonal antibody treatment, the method comprising: (a) mixing a peptide with the sample, wherein the peptide selectively binds to said monoclonal antibody; (b) incubating the peptide-sample mixture, wherein the peptide selectively binds to said monoclonal antibody, forming a peptide-monoclonal antibody complex; and (c) removing the peptide-monoclonal antibody complex from the serum sample.

In another aspect of the present invention there is provided a method of treating a biological sample from a patient for transplantation cross-matching, wherein the patient has been administered a monoclonal antibody treatment, the method comprising the steps of: (a) mixing a peptide with the sample, wherein the peptide selectively binds to said monoclonal antibody; (b) incubating the peptide-sample mixture wherein the peptide selectively binds to said monoclonal antibody, forming a peptide-monoclonal antibody complex; (c) removing the peptide-monoclonal antibody complex from the sample; (d) detecting any monoclonal antibody remaining in the sample; and (e) repeating (a) to (d) if the monoclonal antibody is detected in step (d).

In a further aspect of the present invention there is provided a method of treating a biological sample from a patient for transplantation cross-matching, wherein the patient has been administered rituximab, the method comprising the steps of: (a) mixing a peptide with the sample, wherein the peptide selectively binds to said rituximab; (b) incubating the peptide-sample mixture wherein the peptide binds to said rituximab, forming a peptide-rituximab complex; (c) removing the peptide-rituximab complex from the sample; (d) detecting any rituximab remaining in the sample; and (e) repeating (a) to (d) to remove any remaining rituximab in the sample if necessary.

In yet another aspect of the present invention there is provided a kit for treating a sample from a transplant patient undergoing cross-matching, wherein the patient has been administered a monoclonal antibody treatment, the kit comprising a peptide, wherein the peptide selectively binds the monoclonal antibody and a solid support, wherein the peptide is conjugated to the solid support. The solid support may be a magnetic bead, a non-magnetic bead, a microtiter plate or a tube.

These and other features, aspects and advantages of the present invention will become better understood with reference to the following drawings, description and claims.

### BRIEF DESCRIPTION OF FIGURES

FIG. 1 is an anti-human IgG histogram of normal human serum as analyzed by flow cytometry;

FIG. 2 is an anti-human IgG histogram of normal human serum with 10 μg/mL rituximab as analyzed by flow cytometry;

FIG. 3 is an anti-human IgG histogram of normal human serum with 10 μg/mL rituximab after being treated 3 times with peptide;

FIG. 4 is an anti-human CD20 histogram of normal human serum as analyzed by flow cytometry;

FIG. 5 is an anti-human CD20 histogram of normal human serum with 10 μg/mL rituximab as analyzed by flow cytometry; and

FIG. 6 is an anti-human CD20 histogram of normal human serum with 10 μg/mL rituximab after being treated 3 times with peptide.

### DETAILED DESCRIPTION OF THE INVENTION

While the concept of the present disclosure are illustrated and described in detail in the drawings and the description herein, such an illustration and description are to be considered as exemplary and not restrictive in character, it being understood that only the illustrative embodiments are shown and described and that all changes and modifications that come within the spirit of the disclosure are desired to be protected.

Broadly, the present invention provides methods for depleting a biological sample from a patient to be used for cross-match tissue typing, of at least one monoclonal antibody in which the monoclonal antibody may give a false positive in a cross-matching assay. The method may be used for both pre-transplantation and post-transplantation cross-matching. The monoclonal antibody may be chimeric, humanized, fully human or fully human-mutagenized monoclonal antibodies. Non-limiting examples of monoclonal antibodies contemplated by the present invention, may be anti-CD20, anti-CD3 or anti-CD52 antibodies. Alternatively, the monoclonal antibody may be a synthesized construct where, for example, a protein is attached to an IgG backbone. Non-limiting examples may be abatecept, belatacep or Br3-Fc. Abatecept and belatacep are fusion proteins comprising an immunoglobulin fused to the extracellular domain of CTLA-4. The biological sample, may be, but is not limited to, a serum or plasma sample. The method may comprise providing a peptide or other binding moiety that selectively binds to the monoclonal antibody and contacting the peptide with the sample. After allowing a complex to form between the monoclonal antibody and the peptide, the complex, may be removed from the sample. The sample may then be analyzed to determine the amount of monoclonal antibody remaining in the serum. If the amount is still above the threshold limit for a false positive, then the method may be repeated until the level of monoclonal antibody is below the threshold limit.

The use of a peptide or other binding moiety specific for the monoclonal antibody, particularly if the peptide binds tightly to the antibody, may allow for removal of the monoclonal antibody without removal of other components of the serum such as, alloantibodies. In contrast, methods in the prior art use a polyclonal antibody to remove the monoclonal antibody or a protease treatment to remove the antigen from a cell surface. Both of these methods are often times ineffective in selectively eliminating the monoclonal antibody resulting the false positive caused by the monoclonal still present in the sample. Furthermore, the protease treatment may damage components of the sample complicating subsequent cross-matching assays.

In one embodiment, a peptide or other tightly binding moiety such as, but not limited to, a peptide mimetic that binds with selectivity to the monoclonal antibody may be designed. While peptides will be referred to throughout the description, other antigens besides peptides are contemplated and may be used interchangeably with peptides. Therefore, when used herein, peptide or peptides refers to any moiety, peptide or non-peptide that selectively binds to a monoclonal antibody of interest. Selectively binding peptides and other moieties are those that may bind almost exclusively to the monoclonal antibody and not to other antibodies and components in the serum sample. The peptide may be used as “bait” to fish out any existing monoclonal peptide in the sample. In one illustrative embodiment, once the binding motif to the targeted monoclonal antibody is determined, a peptide that specifically binds to the monoclonal antibody may be synthesized by various methods known in the art. For purification and subsequent application purposes, the synthetic peptide may include tags and/or it be conjugated to a certain linker, for example, a biotinyl group. In another illustrative embodiment, a peptide specific for the monoclonal antibody may be found using phage display peptide libraries. It will be appreciated that there are methods known in the art that would allow the skilled artisan to design a peptide for use in the present method.

In one non-limiting illustrative embodiment, peptides having a high specificity to rituximab (RIT) may be designed. Peptides with homologous sequences to CD20 may be used. Peptides comprising the sequence A(S)NPS may also be used for binding to RIT with a few exceptions. Non-limiting examples of peptides specific for RIT may be NTYNCEPANPSEKNSPSTQYCYSIQ (SEQ ID 1), acPYANPSLc (SEQ ID 2), acPYSNPSLc (SEQ ID 3), WPRWLEN (SEQ ID 4), WPXWLE (SEQ ID 5), QDKLTQWPKWLE (SEQ ID 6) or mixtures thereof, wherein acXXXXc indicates a cyclic peptide wherein the flanking cysteines (c) form an intermolecular disulfide bond. NTYNCEPANPSEKNSPSTQYCYSIQ (SEQ ID 1) may also be a cyclic peptide, with an intermolecular disulfide bond formed between the two cysteine residues while WPRWLEN (SEQ ID 4), WPXWLE (SEQ ID 5) and QDKLTQWPKWLE (SEQ ID 6) are linear peptides. The present invention also contemplates any peptides with conservative substitutions to the above peptides. A conservative substitution is where the size and charge at the amino acid position remains similar after the substitution. Non-limiting examples would be substituting a serine for an alanine or threonine, an aspartic acid for a glutamic acid or a phenylalanine for a tyrosine. Such substitutions are well known to those skilled in the art and may be made to the above peptides without undue experimentation.

After the desired peptide or peptides have been selected they may be immobilized by conjugation onto a solid support such as, but not limited to, beads, the walls of a container such as an Eppendorf or test tube, a pipette tip, a microtiter plate or other solid support. The beads may be magnetic or made of a polymeric material that is non-magnetic. The peptide may be conjugated to the solid support by methods known in the art. For example, the peptide may be covalently attached directly to the solid support. Alternatively, the peptide may be conjugated to the solid support through a binding interaction such as, but not limited to, biotin and streptavidin. A linker may be used to conjugate the peptide to the solid support, but is not required. The peptide may be conjugated to the solid support at either the carboxy- or amino-terminus or alternatively, through a side chain as long as the conjugated peptide is able to bind the monoclonal antibody.

In another embodiment of the present invention, the peptide may be added to and mixed with the biological sample. In one illustrative embodiment the amount of peptide is from about 5 nmol/ml to about 20 nmol/ml. Alternatively, the amount of peptide added may be from about 1 nmol/ml to about 50 nmol/ml. The peptide may or may not be conjugated to the solid support before being mixed with the serum sample. For example, if the peptide is biotinylated, it may be added to the sample and then streptavidin-coated beads may subsequently be added to the serum sample, conjugating the peptide in the serum sample. After adding the peptide to the sample, the mixture may be incubated to allow a peptide-monoclonal antibody complex to form. The sample may be gently agitated during the incubation period to insure optimal interaction between the peptide and the monoclonal antibody. The amount of time required for adequate binding to occur may depend on the concentration of the monoclonal antibody in the serum, the amount of peptide added and/or the binding affinity of the peptide for the monoclonal antibody. The incubation time required for a given sample may be determined empirically by the skilled artisan without undue experimentation.

In a further embodiment of the present invention, the peptide-monoclonal antibody complex may be removed from the sample. In one illustrative embodiment, the peptide may be conjugated to a magnetic bead and may be removed by applying a magnetic field to the sample. The beads may be sequestered on the bottom or side of the sample tube and the treated serum/plasma sample may be removed from the beads, or the beads may be physically removed from the sample. In an alternate illustrative embodiment, the peptide may be conjugated to non-magnetic beads and may be separated out by centrifuging the sample. In yet another illustrative embodiment, the peptide may be conjugated to inner surfaces of a sample tube and the serum sample is introduced into then removed from the tube leaving the peptide-antibody complex on the surfaces (e.g., side) of the tube. Methods of separating a the peptide-antibody complex from the biological sample using beads or physical supports are known in the art and others methods may be used other than the non-limiting examples disclosed herein to illustrate the present invention.

In yet another embodiment of the present invention, the treated serum/plasma sample is analyzed to determine if there is any residual monoclonal antibody remaining in the sample. The amount of monoclonal antibody may be determined by any means known in the art such as, but not limited to, flow cytometry or complement-dependent cytotoxicity. If the amount of monoclonal antibody remaining in the sample is above the threshold level for giving a false positive, the steps of contacting the sample with the peptide, incubating to allow complex formation and removal of the complex may be repeated as many times as necessary to the antibody is depleted from the sample or at least reduced it to a level that no longer interferes with the cross-matching assay. Alternatively, the steps may be repeated until the amount of monoclonal antibody does not decrease after a subsequent treatment. In one illustrative embodiment, the sample is treated from about 2 times to about 5 times. It will be appreciated that the number of times the sample needs to be treated may depend on the amount of monoclonal antibody originally present in the sample, the amount of peptide added and/or the binding affinity of the peptide to the monoclonal antibody. An advantage of the present method is that it may be simply and rapidly performed and therefore treating the sample multiple times is not unduely burdensome.

After the monoclonal antibody is depleted from the sample, the sample may then be used for cross-match tissue typing by any means known in the art such as, but not limited to flow cytometric cross-matching or complement-dependent cytotoxic cross-matching. The depletion of the monoclonal antibody from the serum sample allows for detection of alloantibodies, which if present, indicate whether or not the tissue is compatible with the patient.

In an illustrative embodiment, the amount of monoclonal antibody in the sample may be determined by flow cytometry. To ascertain the removal of monoclonal antibody, a flow cytometry cross match may be performed to compare the depletion treated serum sample containing monoclonal antibody with those not treated serum sample. The donor cells expressing the antigens that are recognized by the monoclonal antibody may be incubated with a patient sample treated or not treated using the aforementioned depletion methods. After the washing step, a fluorescein isothiocyanate (FITC)-conjugated anti-human IgG may be applied to the mixture of donor cell and patient serum/plasma sample treated or not treated with depletion methods aforementioned. If the monoclonal antibody in the sample serum is successfully removed, less FITC-conjugated anti-human IgG antibody may be recognized on the donor cells. Alternatively, the donor cells expressing the antigens that are recognized by the monoclonal antibody may be incubated with a patient sample treated or not treated with aforementioned depletion methods. After the washing step, a FITC-conjugated antibody against human lymphocyte antigen may be applied to the mixture of donor cells and patient sample treated or not treated with depletion methods aforementioned. If the monoclonal antibody in the sample is successfully removed, more FITC-conjugated antibody against the human lymphocyte antigen will be recognized on the donor cells.

In another illustrative embodiment, the monoclonal antibody is rituximab and the threshold or cutoff values for B-cell flow cytometic cross-matching is established by each individual laboratory depending on specific procedure or instrument variations.

The present invention further provides a kit for treating a sample from a patient for pre-transplantation cross-matching where the patient has been administered a monoclonal antibody. The kit may comprise a peptide, where the peptide selectively binds the monoclonal antibody, allowing for removal of the monoclonal antibody from the sample without removing significant amounts, if any, of other components of the sample such as, but not limited to, alloantibodies. The kit may further comprise a solid support to which the peptide may be conjugated. The solid support may be, but not limited to, beads, the walls of a container such as an Eppendorf or test tube, a pipette tip, a microtiter plate or other solid support. The beads may be magnetic or made of a polymeric material that is non-magnetic. The peptide may be conjugated to the solid support by methods known in the art. For example, the peptide may be covalently attached to the solid support. Alternatively, the peptide may be conjugated to the solid support through a binding interaction such as, but not limited to, biotin and streptavidin. A linker may be used to conjugate the peptide to the solid support, but is not required. The peptide may be conjugated to the solid support at either the carboxy- or amino-terminus or alternatively, through a side chain as long as the conjugated peptide is able to bind the monoclonal antibody.

The kit may further comprise components required to determine whether or not the monoclonal antibody has been depleted from the sample. For example, antibodies directed toward the monoclonal antibody may be included in the kit.

### EXAMPLES

The synthesis of the cyclic CD20 peptide 245-98F5 was carried out as follows. NTYNCEPANPSEKNSPSTQYCYSIQ-K(PEG2-Biotin)-G (SEQ ID 1), with an intermolecular S—S bond was started using Fmoc chemistry, Mtt-protected lysine26 and Fmoc-Gly-wang resin manually. After the Lys (Mtt)²⁶ was coupled, the peptide-resin bearing the Mtt protected lysine was treated with TFA/TIS/DCM to remove the Mtt group. The N-biotinyl-NH-(PEG)₂-COOH was conjugated to the ε-amine group of the lysine. The continuing synthesis of the peptide was carried out on an ABI 433A peptide synthesizer (Applied Biosystems Inc.), using Fmoc chemistry and tritl-protected Cys. The cleavage of the peptide from the resin and side-chain deprotection was accomplished using a TFA cocktail reagent and the crude peptide was precipitated with ether and lyophilized. The disulfide bond formation of the peptide was done in aqueous DMSO at PH 7.5. The final peptide was purified by preparative reverse-phase HPLC and was characterized by analytical HPLC and molecular weight analysis by mass spectroscopy.

The purified peptide was reconstituted with 0.1% bovine serum albumen (BSA). Twenty-five nMoles of solublized peptide were added to 16.75×10⁶ washed Dynal M-280 super-paramagnetic polystyrene beads (Invitrogen, Carlsbad, Calif.). The peptide and beads, precoated with streptavidin covalently attached to the hydrophobic surface, were incubated for 30 minutes at room temperature in a 12×75 polystyrene tube. Experiments demonstrated no difference in absorption results with either twice or half as much peptide for the same amount of beads, or a change in the incubation time from 15 minutes to 4 hours. Excess peptide solution was removed by adhering the beads to a magnet for 3 minutes with removal and discarding of the supernatant. This step was repeated three times. The peptide-coated beads were stored in the refrigerator at 4° C. until needed. For absorption of rituximab containing serum, the peptide-coated beads were resuspended in 2.5 mL of BSA solution. One hundred μl of the bead suspension was added to a 12×75 polystyrene tube. The supernatant was eliminated by adhering the beads to a magnet for 3 minutes. Normal human serum containing added rituximab, and appropriate controls, were then added to the beads and incubate for 15 minutes on a rocker at room temperature. The beads were removed by adherence to a magnet for 3 minutes. The serum was retained and further absorbed as needed by repeating the above steps until rituximab was removed. The final absorbed serum was now available for routine cross-matching techniques such as flow cytometry, or cytotoxicity assays.

For flow cytometry cross-matching, 250,000 donor splenocytes were incubated with 25 μl of test serum on ice. The cells were washed with BSA solution containing sodium azide. Goat anti-human IgG F(ab′)₂ FITC-conjugated antibody (Jackson ImmunoResearch, West Grove, Pa.) and anti-human CD20 PE-conjugated (BD Biosciences, San Jose, Calif.) were added. After incubation, washing, and fixation of the sample with paraformaldehyde, the samples were analyzed by flow cytometry.

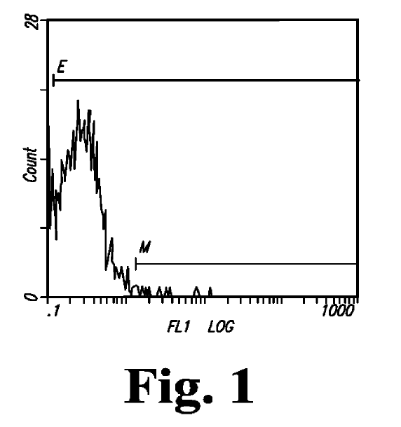

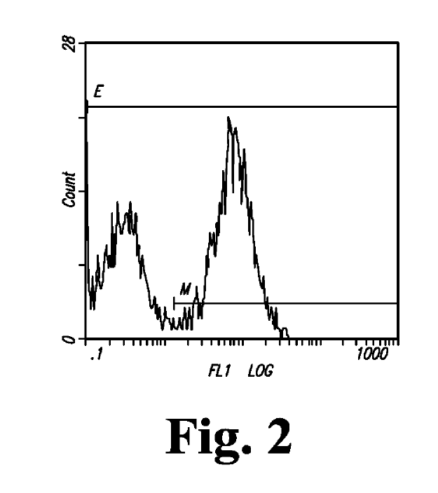

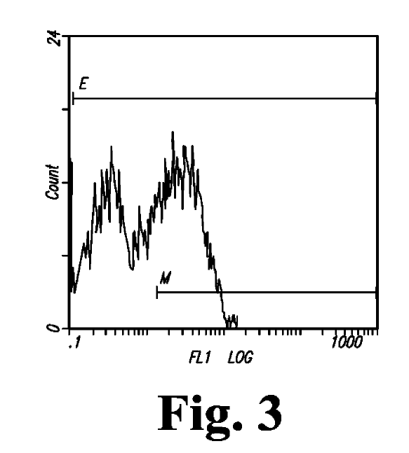

A representative experiment is shown in FIGS. 1-6. Median channel of log green and log red fluorescence and percent positive cells were compared for sera before and after treatment and with or without added rituximab. FIG. 1 shows the negative control of flow cytometric cross match between donor spleen cells and normal human serum that does not contain any alloantibody or chimeric rituximab monoclonal antibody. The fluorescent antibody used in FIG. 1 was FITC labeled anti human IgG1, which recognizes the Fc portion of the alloantibody as well as the Fc portion of rituximab. The lack of recognizable Fc portion in the cross match serum gave a negative reading on fluorescence. FIG. 2 shows flow cytometric cross match between donor spleen cells and normal human serum that contains 10 μg/mL of rituximab but no alloantibody. The presence of rituximab's Fc portion gave a prominent positive peak comparable to alloantibody positive control (data not shown), a result of the recognition of FITC labeled anti-human IgG1. FIG. 3 shows a cross match between the donor spleen cells and normal human serum that contains 10 μg/mL of rituximab with no alloantibody, but after 3 depletion treatments described herein. The reduced rituximab in the serum was indicated by a smaller number of fluorescence labeled cells, recognized by FITC labeled anti-human IgG1. The channel of positive peak shifted to the left of the histogram, and the intensity decreased in FIG. 3 compared to that of FIG. 2. Further analysis showed the median channel of log green fluorescence was 0.280 for NHS, 4.44 for NHS containing 10 μg/mL of rituximab before absorption and 1.32 after 3 treatments. This indicated that in FIG. 3 less rituximab was bound to the cells and was identified by binding of the goat anti-human IgG F(ab′)₂ FITC conjugated antibody.

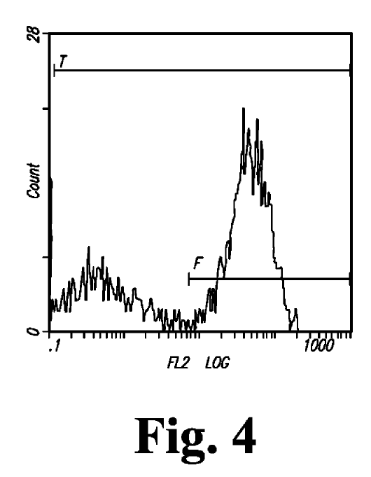

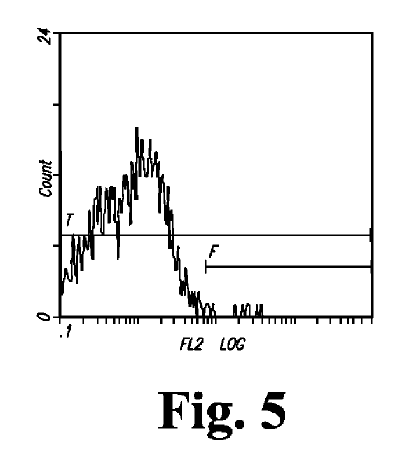

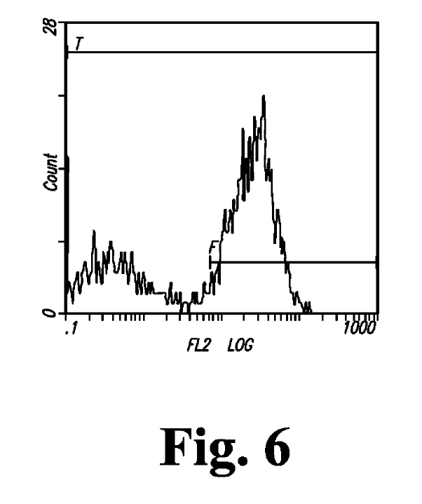

FIG. 4 is a negative control of flow cytometric cross match between donor spleen cells and normal human serum that did not contain any alloantibody or chimeric monoclonal antibody. The fluorescent antibody used in FIG. 4 was phycoerytherin (PE) labeled anti-human CD20. In the absence of rituximab to compete with fluorescence anti-CD20, a positive binding of PE labeled anti-CD20 was detected in the donor cells, indicated by the positive peak in the histogram. FIG. 5 shows flow cytometric cross match between donor spleen cells and normal human serum that contains 10 μg/mL of rituximab but no alloantibody. In the presence of rituximab that specifically binds to CD20 on the donor spleen cells, the CD20 were blocked; therefore almost no CD20-PE labeled cells were detected, indicated by the disappearance of positive peak in the histogram. FIG. 6 shows a cross-match between the donor spleen cells and normal human serum that contains 10 μg/mL of rituximab with no alloantibody, but after 3 depletion treatments described herein. The reduced rituximab in the serum was indicated by an increased number of fluorescence labeled cells detected by PE anti-human CD20. The channel of positive peak re-appeared in the histogram, and the intensity was comparable to the negative control.

Further analysis showed that percent positive PE fluorescence anti-human CD20 was 56.7 in NHS, 0.84 for NHS containing 10 μg/mL of rituximab before absorption, and 56.7 after 3 treatments. Median channel of PE fluorescence anti-human CD20 was 32.9 for NHS, 0.903 for NHS containing 10 μg/mL of rituximab before absorption, and 18.6 after 3 treatments. This indicated that rituximab interference had been reduced.

It should be understood, of course, that the foregoing relates to exemplary embodiments of the invention and that modifications may be made without departing from the spirit and scope of the invention as set forth in the following claims. It should be understood, of course, that the foregoing relates to exemplary embodiments of the invention and that modifications may be made without departing from the spirit and scope of the invention as set forth in the following claims.

What is claimed is:

1. A method of treating a biological sample from a patient for transplantation cross-matching, wherein the patient had been administered a monoclonal antibody treatment, the method comprising:
(a) mixing a peptide with the biological sample wherein the peptide selectively binds to said monoclonal antibody;
(b) incubating the peptide-sample mixture wherein the peptide selectively binds to said monoclonal antibody, forming a peptide-monoclonal antibody complex; and
(c) removing the peptide-monoclonal antibody complex from the biological sample.

2. The method of claim 1, further comprising the steps of:
(d) detecting any monoclonal antibody remaining in the serum sample after step (c); and
(e) repeating (a) to (c) if the monoclonal antibody is detected in step (d).

3. The method of claim 2 wherein the monoclonal antibody is detected by flow cytometry.

4. The method of any one or more of claim 1 wherein the peptide is conjugated onto a solid support.

5. The method of claim 4 wherein the solid support is a magnetic or non-magnetic bead.

6. The method of claim 4 wherein the solid support comprise a surface of a tube.

7. The method of claim 4 wherein the peptide is biotinylated and the solid support is coated with strepavidin.

8. The method of claim 1 wherein the monoclonal antibody is a chimeric, humanized, fully human or fully human-mutagenized monoclonal antibody.

9. The method of claim 1 wherein the monoclonal antibody is an anti-CD20 antibody, an anti-CD3 antibody or an anti-CD52 antibody.

10. The method of claim 9 wherein the monoclonal antibody is an anti-CD20 antibody and the peptide is NTYNCEPANPSEKNSPSTQYCYSIQ, acPYANPSLc, acPYSNPSLc, WPRWLEN, WPXWLE, QDKLTQWPKWLE or combinations thereof.

11. A kit for carrying out the method of claim 1, the kit comprising:
a peptide wherein the peptide selectively binds the monoclonal antibody; and
a solid support wherein the peptide is conjugated to the solid support.

12. The kit of claim 11 wherein the solid support is a magnetic bead, a non-magnetic bead, a microtiter plate or tube.

13. A method of treating a biological sample from a patient for transplantation cross-matching, wherein the patient had been administered a monoclonal antibody treatment comprising an anti-CD20 antibody, an anti-CD3 antibody or an anti-CD52 antibody, the method comprising:
(a) mixing a peptide with the biological sample wherein the peptide selectively binds to said monoclonal antibody;
(b) incubating the peptide-sample mixture wherein the peptide selectively binds to said monoclonal antibody, forming a peptide-monoclonal antibody complex; and
(c) removing the peptide-monoclonal antibody complex from the biological sample.

14. The method of claim 13, further comprising the steps of:
(d) detecting any monoclonal antibody remaining in the serum sample after step (c); and
(e) repeating (a) to (c) if the monoclonal antibody is detected in step (d).

15. The method of claim 14 wherein the monoclonal antibody is detected by flow cytometry.

16. The method of any one or more of claim 13 wherein the peptide is conjugated onto a solid support.

17. The method of claim 13 wherein the monoclonal antibody is a chimeric, humanized, fully human or fully human-mutagenized monoclonal antibody.

18. A method of treating a biological sample from a patient for transplantation cross-matching, wherein the patient had been administered a monoclonal antibody treatment comprising an anti-CD20 antibody, the method comprising:
(a) mixing a peptide with the biological sample wherein the peptide selectively binds to said monoclonal antibody;
(b) incubating the peptide-sample mixture wherein the peptide selectively binds to said monoclonal antibody, forming a peptide-monoclonal antibody complex;
(c) removing the peptide-monoclonal antibody complex from the biological sample;
(d) detecting any monoclonal antibody remaining in the serum sample after step (c); and
(e) repeating (a) to (c) if the monoclonal antibody is detected in step (d).

19. The method of claim 18 wherein the peptide is NTYNCEPANPSEKNSPSTQYCYSIQ, acPYANPSLc, acPYSNPSLc, WPRWLEN, WPXWLE, QDKLTQWPKWLE or combinations thereof.

20. The method of claim 18 wherein the monoclonal antibody is detected by flow cytometry.
